(19) United States
(12) Patent Application Publication
(10) Pub. No.: US 2016/0040171 A1
(43) Pub. Date: Feb. 11, 2016

(54) METHOD TO PRODUCE HYDROCARBON FROM C-1 SUBSTRATE

(71) Applicants: WILLIAM MARSH RICE UNIVERSITY, Houston, TX (US); TECHNOLOGY HOLDING LLC, Salt Lake City, UT (US)

(72) Inventors: Ka-Yiu SAN, Houston, TX (US); George BENNETT, Houston, TX (US); Mukund KARANJIKAR, West Valley City, UT (US)

(21) Appl. No.: 14/919,911

(22) Filed: Oct. 22, 2015

Related U.S. Application Data
(63) Continuation of application No. PCT/US14/34861, filed on Apr. 22, 2014.
(60) Provisional application No. 61/814,626, filed on Apr. 22, 2013.

(51) Int. Cl. C12N 15/52; C12P 5/00; C12P 7/62; C12P 7/64

(52) U.S. Cl. CPC C12N 15/52; C12P 7/6409; C12P 5/00; C12P 7/62

(57) ABSTRACT

The disclosure relates to biological methods of making a hydrocarbon feedstock wherein one-carbon substrates are converted into useful chemicals and fuels. Particularly, genetically engineered bacteria are used to make C4-C10 fatty acids or derivatives from one-carbon substrates such as methanol and carbon dioxide.

### PRIOR RELATED APPLICATIONS

This application claims priority to U.S. Ser. No. 61/814,626, filed Apr. 22, 2013, and PCT Application No. PCT/US14/34861, filed Apr. 22, 2014, each of which is expressly incorporated by reference herein in its entirety for all purposes.

### FEDERALLY SPONSORED RESEARCH STATEMENT

Not applicable.

### FIELD OF THE DISCLOSURE

The disclosure generally relates to biological methods of making hydrocarbon feedstocks.

### BACKGROUND OF THE DISCLOSURE

Four to ten carbon alkanes have many uses in our society, particularly as fuels and as feedstock for more complex chemicals. Most, however, is produced from petroleum, a dwindling reserve whose use creates significant ecological impact.

Normal butane (C4), for example, is mainly used for gasoline blending, as a fuel gas, either alone or in a mixture with propane, and as a feedstock for the manufacture of ethylene and butadiene, a key ingredient of synthetic rubber. Isobutane is primarily used by refineries to enhance the octane content of motor gasoline.

Butane is also used as lighter fuel for a common lighter or butane torch and is sold bottled as a fuel for cooking and camping. Cordless hair irons are usually powered by butane cartridges.

In industry, hexanes (C6) are used in the formulation of glues for shoes, leather products, and roofing. They are also used to extract cooking oils from seeds, for cleansing and degreasing a variety of items, and in textile manufacturing. A typical laboratory use of hexanes is to extract oil and grease contaminants from water and soil for analysis. Since hexane cannot be easily deprotonated, it is used in the laboratory for reactions that involve very strong bases, such as the preparation of organolithiums, e.g. butyllithiums, which are typically supplied as a hexane solution. In many applications (especially pharmaceutical), the use of n-hexane is being phased out due to its long term toxicity, and often replaced by n-heptane, which will not form the toxic metabolite hexane-2,5-dione.

Octanes (C8) became well known in American popular culture in the mid- and late-sixties, when gasoline companies boasted of “high octane” levels in their gasoline advertisements. Thus, it too is useful in fuels. Decane (C10) undergoes combustion reactions in a similar fashion to other alkanes.

Thus, we can see that there are many important uses for low carbon number alkanes and the demand for C4-10 alkanes is not expected to diminish any time soon. Yet as products of petroleum refining, the production of such alkanes contributes significantly to environmental degradation, and as our hydrocarbon resources continue to dwindle in availability, the alkanes can only be expected to increase in price over the long term.

Thus, what are needed in the art are biological sources for these important alkanes, and microbial production is being investigated in that regard. Unfortunately, not many bacteria make butane or hexane, at least not in significant amounts, and some of the bacteria that do are obligate anaerobes, which are difficult and expensive to culture.

Professor David Mullin, and his team have discovered a new bacteria, called Tu-103, a butane-producing bacteria that lives on glycerol—a byproduct of biodiesel synthesis, or on cellulose—a waste product in abundant supply from e.g., old newspapers. The microbe is unique because it can do this in the presence of oxygen, unlike some other types of bacterium, which means less expensive production techniques would be required than for most obligate anaerobes. However, little is known about this bacteria because details are being kept as a trade secret, and future patents may also prevent its use.

What are needed in the art are additional methods of make C4-10 alkanes using microbes.

### SUMMARY OF THE DISCLOSURE

This disclosure focuses on the microbial conversion of one-carbon substrates such as methane, methanol, formate, carbon dioxide and carbon monoxide to useful chemicals and fuels. These 1-carbon substrates are abundant in nature and thus provide good and inexpensive source of carbon substrate that can be converted to at least C4⁺ acids, and from there to a wide variety of useful molecules.

The disclosure provides a rational design of microbial metabolic pathways to first convert methanol and carbon dioxide to short chain length organic acids. Preferably, such microbes are aerobes or even more preferred are facultative anaerobes.

The second step is the electrochemical conversion of organic acids to hydrocarbon. Hydrogen and carbon dioxide formed during the electrochemical process can be recycled back to the fermentator to provide additional carbon substrate as well as reducing equivalents.

FIG. 1 shows the microbial conversion of methanol and carbon dioxide first to butyrate and to hexanoate followed by electrochemical conversion to hydrocarbon. The enzyme(s) associated with the various reactions are listed in the enzyme Table A and B below.

Enzyme Table A shows the enzymes whose expression must typically be added to bacteria, with the possible exception of the enzyme of reaction 6 or any other enzyme that is endogenous in the chosen host species. Enzymes up to reaction 11 must be added to make butyrate, and up to 13 for hexanoate. Additional enzymes can be obtained by database search or homology search with a particular sequence, and several protein homologs are shown in Table B.

TABLE A
Enzymes

| | | | Exemplary Acc. Nos. species |
|---|---|---|---|
| Rxn | Enzyme name | EC number | (see additional homologs in Table B) |
| 1 | methanol | EC 1.1.1.244 | EU548062 Methyloversatilis universalis FAM5 |
| | dehydrogenase | EC 1.1.99.37 | M65004.1 Bacillus methanolicus |
| | | | P15279 (UniPort) Methylobacterium organophilum |
| | | | C5MRT8 (UniProt) Mycobacterium sp. DSM 3803 |
| 2 | Spontaneous reaction | No enzyme | / |
| | | needed | |
| 3 | methylene | EC 2.1.1.13 | CAA58474 (Emb); Q42699 (UniProt) Catharanthus roseus |
| | tetrahydrofolate | | AT5G17920 (MetaCyc, tair); AAC50037.1 Arabidopsis: |
| | reductase | | G-7121 (MetaCyc); AAF74983.1 Solanum tuberosum |
| 4 | formaldehyde | EC 1.2.1.46 | P46154 (UniProt) Pseudomonas putida |
| | dehydrogenase | EC 1.2.7.5 | CAA57973 Arabidopsis thaliana |
| | | EC 1.2.99.4 | |
| 5 | formate dehydrogenase | EC 1.2.1.2 | O13437 (UniProt) Candida boidinii |
| | | EC 1.2.1.43 | Q845T0 (UniProt) Ancylobacter aquaticus |
| | | | MTU73807 Moorella thermoacetica |
| 6 | carbon monoxide | EC 1.2.7.4 | Q2RH19 (UniProt) Moorella thermoacetica |
| | dehydrogenase-acetyl- | EC 1.2.99.2 | U80806 (Embl) Hydrogenophaga pseudoflava |
| | CoA synthase | EC 2.3.1.169 | |
| 7 | thiolase or | EC 2.3.1.9 | Q46939 (UniProt) Escherichia coli |
| | acetoacetyl-CoA | EC 2.3.1.16 | F7V739 (UniProt) Clostridium |
| | synthase | EC 2.3.1.194 | Q8S4Y1 (UniProt) Arabidopsis thaliana |
| 8 | hydroxybutyryl-CoA | EC 1.1.1.157 | F0K419 (UniProt) Clostridium acetobutylicum |
| | dehydrogenase | EC 1.1.1.35 | XP_003190756 Aspergillus oryzae |
| 9 | crotonase or Enoyl- | EC 4.2.1.55 | C8TRC5 (UniProt) Escherichia coli |
| | CoA hydratase- | EC 4.2.1.17 | P07896 (UniProt) Rattus norvegicus |
| | isomerase or | | AAN72209.1 Arabidopsis |
| | Peroxisomal | | AT1G60550.1 (TAIR) Arabidopsis |
| | bifunctional enzyme | | AEE74468 (MFP2) Arabidopsis |
| 10 | butyryl-CoA | EC 1.3.99.2 | YP_001309209 Clostridium beijerinckii |
| | dehydrogenase | EC 1.3.1.38 | A8FZ69 (UniProt) Shewanella sediminis: |
| | or | EC 1.3.1.44 | AAA95968.1 (beta-hydroxybutyryl-coenzyme A (CoA) |
| | trans-enoyl-CoA | | dehydrogenase, crotonase, and butyryl-CoA |
| | reductase | | dehydrogenase) C. acetobutylicum |
| | | | WP_002669849: Treponema denticola |
| | | | AAW66853: Euglena gracilis |
| | | | U72144.2: 1-cyclohexenylcarbonyl CoA reductase |
| | | | Streptomyces collinus |
| | | | AAA92890.1: crotonyl CoA reductase Streptomyces |
| | | | collinus |
| 11 | phosphotransbutyrylase | EC 2.3.1.19 | L14744 Clostridium acetobutylicum |
| | and | EC 2.7.2.7 | A7FZ19 (UniProt) Clostridium botulinum |
| | butyrate kinase | EC 2.7.2.14 | Q05624 (UniProt), L04468.1 Clostridium beijerinckii |
| | | | Q97II1 (UniProt) Clostridium acetobutylicum buk2 |
| | | | NP_560986.1 Clostridium PBT |
| 12 | Enzymes 7-10 | EC 1.3.8.1 | B1L0Z9 (UniProt) Butyryl-CoA dehydrogenase |
| | | | Clostridium botulinum |
| | | | B1IN19 (UniProt) Clostridium botulinum: |
| | | | see also rxn 7-10 |
| 13 | phosphotransbutyrylase | EC 2.3.1.19 | A7FZ19 (UniProt) Clostridium botulinum |
| | and butyrate kinase or | | Q05624, L04468 Clostridium beijerinckii |
| | homologs with | | |
| | specificity for longer | | |
| | chain acids | | |

TABLE B
Additional Enzymes

Homologs for ACB73286.1 Methanol/alcohol dehydrogenase
Methyloversatilis universalis Fam5
WP_020164279.1 Rhodocyclaceae bacterium RZ94 (497/523 = 95%
amino acid identity)
ACB73290.1 Burkholderiales bacterium RZ18-153 (496/522 = 95%)
YP_934475.1 Azoarcus sp. BH72 (433/523 = 83%)
YP_001019673.1 Methylibium petroleiphilum PM1 (430/523 = 82%)
Homologs to Q42699 Cobalamin-independent methionine synthase
isozyme, Catharanthus roseus
ABI35986.1 Orobanche ramose (701/765 = 92%)
AGT40326.1 Nicotiana attenuate (704/765 = 92%)
ABS01352.1 Carica papaya (698/765 = 91%)
XP_002525709.1 Ricinus communis (696/765 = 91%)
Homologs to P46154 Glutathione-independent formaldehyde
dehydrogenase, Pseudomonas putida
ACP17957.1 Pseudomonas nitroreducens (393/399 = 98%)
WP_020481043.1 Pseudomonas fuscovaginae (388/399 = 97%)
WP_004373711.1 Pseudomonas mendocina (388/399 = 97%)
YP_004476269.1 Pseudomonas fulva 12-X (384/399 = 96%)
Homologs to CAA57973 formaldehyde dehydrogenase, Arabidopsis
Eutrema salsugineum: XP_006403162.1 (366/379 = 97%)
Brassica oleracea var. botrytis: AFP72379.1 (365/378 = 97%)
Cucumis sativus: XP_004140582.1 (349/375 = 93%)
Prunus persica: XP_007199930.1 (350/377 = 93%)
Homologs to O13437 NAD-dependent formate dehydrogenase,
Candida boidinii
Candida methylica: CAA57036.1 (363/364 = 99%)
Komagataella pastoris GS115: XP_002493171.1 (314/364 = 86%)
Ogataea parapolymorpha DL-1: ESW96524.1 (300/359 = 84%)
Candida boidinii: ABE69165.2 (351/364 = 96%)
Ogataea parapolymorpha DL-1: ESW96524.1 (300/359 = 84%)
Homologs to AAD00361.1 CutM carbon monoxide dehydrogenase,
Hydrogenophaga pseudoflava
Limnohabitans sp. Rim47: WP_019429897.1 (247/287 = 86%)
Betaproteobacteria bacterium MOLA814: WP_023473753.1
(215/286 = 75%)
Homologs to acetyl-CoA acetyltransferase Q46939 Escherichia coli
Shigella boydii: WP_000656006.1 (391/393 = 99%)
Enterobacteriaceae; WP_000656043.1 (390/393 = 99%)
Shigella flexneri: WP_000656024.1 (390/393 = 99%)
Homologs to F0K419 3-hydroxybutyryl-CoA dehydrogenase,
Clostridium acetobutylicum
Clostridium botulinum: WP_003364514.1 (240/282 = 85%)
Clostridium arbusti: WP_010237079.1 (237/282 = 84%)
Desulfobacter curvatus: WP_020585308.1 (208/280 = 74%)
Homologs for C8TRC5 Enoyl-CoA hydratase-isomerase, E. coli
Shigella flexneri: WP_001292336.1 (250/255 = 98%)
Shigella boydii: WP_001292335.1 (250/255 = 98%)
Enterobacter cloacae subsp. dissolvens SDM: YP_006477509.1
(217/255 = 85%)
Homologs to P07896: Peroxisomal bifunctional enzyme: Enoyl-CoA
hydratase/3,2-trans-enoyl-CoA isomerase and 3-hydroxyacyl-CoA
dehydrogenase, Rattus norvegicus
Mus musculus: NP_076226.2 (662/722 = 92%)
Peromyscus maniculatus bairdii: XP_006987793.1 (624/722 = 86%)
Cricetulus griseus: XP_003504267. (619/722 = 86%)
Homo sapiens: NP_001957.2 (562/723 = 78%)
Homologs to NP_560986: phosphotransbutyrylase [Clostridium
perfringens str. 13].
Clostridium perfringens: WP_003479865.1 143/148 (97%)
Fervidicella metallireducens AeB: EYE87185.1 69/137 (50%)

The overall reactions for the microbial conversion of methanol and carbon dioxide to butyrate and hexanoate are:

$$3 \frac{1}{3} {CH}_{4} O + 2 {CO}_{2} + ADP + \frac{1}{3} {H}_{2} O + {P}_{i} \to {C}_{4} {H}_{8} {O}_{2} + 1 \frac{1}{3} {CO}_{2} + 3 {H}_{2} O + ATP$$

$$5 \frac{1}{3} {CH}_{4} O + 3 {CO}_{2} + ADP + {P}_{i} \to {C}_{6} {H}_{12} {O}_{2} + 2 \frac{1}{3} {CO}_{2} + 4 \frac{2}{3} {H}_{2} O + ATP$$

The reactions for the electrochemical conversion for the formation of hexane, octane and decane are:

2C₄H₈O₂→C₆H₁₄+2CO₂+H₂+2e−

2C₆H₁₂O₂→C₁₀H₂₂+2CO₂+H₂+2e−

C₆H₁₂O₂+C₄H₈O₂→C₈H₁₈+2CO₂+H₂+2e−

The overall reaction for the methanol and carbon dioxide to hexane and decane are:

$$6 \frac{2}{3} {CH}_{4} O + 4 {CO}_{2} + 2 ADP + {P}_{i} + \frac{2}{3} {H}_{2} O \to {C}_{6} {H}_{14} + {H}_{2} + 4 \frac{2}{3} {CO}_{2} + 6 {H}_{2} O + 2 ATP + 2 {e}^{-}$$

$$10 \frac{2}{3} {CH}_{4} O + 6 {CO}_{2} + 2 ADP + {P}_{i} \to {C}_{10} {H}_{22} + {H}_{2} + 6 \frac{2}{3} {CO}_{2} + 9 \frac{1}{3} {H}_{2} O + 2 ATP + 2 {e}^{-}$$

Initial cloning experiments are proceeding in E. coli for convenience since most of the above genes are already available in plasmids suitable for expression in E. coli, but the addition of genes to bacteria is of nearly universal applicability, so it will be possible to use a wide variety of organisms with the selection of suitable vectors for same. Furthermore, a number of databases include vector information and/or a repository of vectors. See e.g., Addgene.org which provides both a repository and a searchable database allowing vectors to be easily located and obtained from colleagues. See also Plasmid Information Database (PlasmID) and DNASU having over 191,000 plasmids. A collection of cloning vectors of E. coli is also kept at the National Institute of Genetics as a resource for the biological research community. Furthermore, vectors (including particular ORFS therein) are usually available from colleagues.

The enzymes can be added to the genome or on expression vectors, as desired. Preferably, multiple enzymes are expressed in one vector or multiple enzymes can be combined into one operon by adding the needed signals between coding regions. Further improvements can be had by overexpressing one or more, or even all of the enzymes, e.g., by adding extra copies to the cell via plasmid or other vector. Initial experiments will employ expression plasmids hosting 3 or more ORFs for convenience, but it may be preferred to insert operons or individual genes into the genome for stability reasons.

Still further improvements in yield can be had be removing competing pathways, such as those pathways for making lactate or acetate or ethanol, and it is already well known in the art how to reduce or knockout these pathways. Our own lab has several patent applications addressing such improvements, and such hosts will also make suitable starting materials since they are already available.

The advantages of the innovation are:

1. Microbial production of acids conserves redox and forms ATP relative to production of alcohols and allows robust cell performance.

2. Use of methanol as substrate provides reductant for formation of longer chain length acids.

3. Use of carbon dioxide as a co-substrate.

4. Electro decarboxylation is efficient and forms longer chain hydrocarbon than individual fatty acid molecules while producing hydrogen.

The invention includes one or more of the following embodiments, in any combination thereof:

```
A genetically engineered bacteria that can convert methanol and carbon
dioxide to C4+ fatty acids.
A genetically engineered bacteria, expressing each of:
  methanol dehydrogenase;
  methylene tetrahydrofolate reductase;
  formaldehyde dehydrogenase;
  formate dehydrogenase;
  carbon monoxide dehydrogenase-acetyl-CoA synthase;
  thiolase or acetoacetyl-CoA synthase;
  hydroxybutyryl-CoA dehydrogenase;
  crotonase or Enoyl-CoA hydratase-isomerase or peroxisomal
  bifunctional enzyme;
  butyryl-CoA dehydrogenase or trans-enoyl-CoA reductase; and
  phosphotransbutyrylase and butyrate kinase;
said bacteria able to convert methanol and carbon dioxide to ≧C4 fatty acids.
A bacteria wherein said bacteria is i) an acetogenic bacteria or ii) a
Clostridium, Clostridium thermocellum, Clostridium ljungdahlii,
Clostridium thermoautotrophicum, or Clostridium tyrobutyricum or iii)
Butyrobacterium, Moorella thermoacetica, Sporomusa,
Thermacetogenium phaeum, Acetogenium kivui, Acetobacterium woodii,
or Eubacterium.
Any bacteria herein described.
A bacteria wherein one or more of said enzymes are selected from those
listed in Table A or B.
A method for the bioproduction of fatty acids or their derivatives, said
method comprising:
  growing the bacteria as herein described in a culture medium providing
  a C1 carbon source;
  conversion of said C1 carbon source to fatty acids of chain length
  ≧C4; and
  isolation of said fatty acids.
A method for the bioproduction of fatty acids from methanol and carbon
dioxide by growing the bacteria herein described in a medium
comprising methanol and carbon dioxide until fatty acids are produced,
and harvesting said produced fatty acids.
A method for the bioproduction of hydrocarbons, said method comprising:
  growing the bacteria as described herein in a culture medium providing
  a C1 carbon source;
  conversion of said C1 carbon source to fatty acids of chain length ≧C4,
  electrochemical conversion of said fatty acids to hydrocarbons and H₂
  and CO₂.
A method as herein described, further comprising converting said fatty
acids to esters.
A method as herein described, wherein said H₂ and CO₂ are recycled to said
culture medium.
A method as herein described, wherein said fatty acids are converted to
esters.
A method as herein described, wherein said fatty acids are converted to
hydrocarbons.
A method as herein described, followed by electrochemical conversion of
said fatty acids to hydrocarbons and H₂ and CO₂.
A method as herein described, wherein said H₂ and CO₂ are recycled to
said culture medium.
A system for producing fatty acids or hydrocarbons, said system comprising
a bioreactor containing a growth medium, and a bacteria as herein
described, said bioreactor fluidly coupled to separator for separating said
bacteria and any secreted fatty acids from bacteria and growth medium,
and a return flow to said bioreactor for returning said bacteria and said
growth medium to said bioreactor.
A system for producing fatty acids or hydrocarbons, said system comprising
a bioreactor containing a growth medium, and a bacteria as herein
described, said bioreactor fluidly coupled to separator for separating said
bacteria and any secreted fatty acids from bacteria and growth medium.
A system further comprising a first flow return pipe fluidly coupling said
bioreactor and said separator for returning said bacteria and/or growth
medium to said bioreactor.
A system wherein said fatty acids are secreted and wherein said separator
skims fatty acids from a surface of said growth medium, and said first flow
return pipe returns the growth medium to said bioreactor.
A system further comprising a reactor fluidly coupled to said separator, said
reactor for electrochemical conversion of said fatty acids to hydrocarbons
and H2 and CO2, preferably also comprising a second flow return pipe
fluidly coupling said reactor and said bioreactor for returning said CO2 to
said bioreactor.
```

### DESCRIPTION OF DRAWINGS

FIG. 1 shows the pathway needed to convert methanol and carbon dioxide to short chain alkanes.

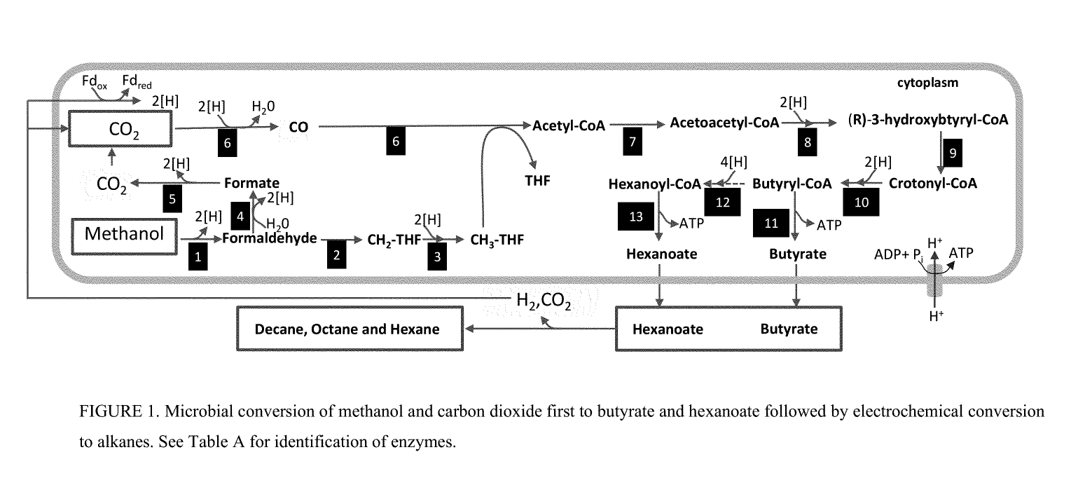

FIG. 2 shows an integrated process combining fermentation tanks with collection of fatty acids and chemical conversion to hydrocarbons.

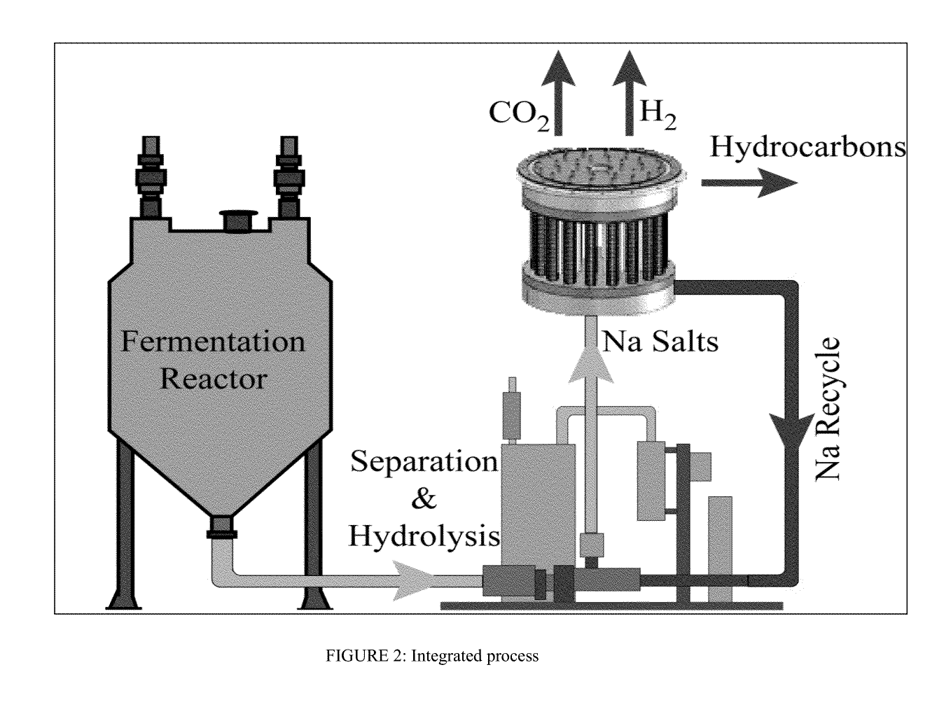

FIG. 3 shows the “Wood-Ljungdahl” or “reductive acetyl-CoA” pathway from certain bacteria and archaea, wherein carbon dioxide is reduced to carbon monoxide, which is then converted to acetyl coenzyme A.

### DETAILED DESCRIPTION

The disclosure relates to bacteria genetically engineered to express a) methanol dehydrogenase, b) methylene tetrahydrofolate reductase, c) formaldehyde dehydrogenase, d) formate dehydrogenase, e) carbon monoxide dehydrogenase or acetyl-CoA synthase, f) thiolase or acetoacetyl-CoA synthase, g) hydroxybutyryl-CoA dehydrogenase, h) crotonase or Enoyl-CoA hydratase-isomerase or Peroxisomal bifunctional enzyme, i) butyryl-CoA dehydrogenase or trans-enoyl-CoA reductase, j) phosphotransbutyrylase and butyrate kinase and k) phosphotransbutyrylase and butyrate kinase or homologs with specificity for longer chain acids.

The disclosure also relates to methods of making C4-10 fatty acids or derivatives therefrom from methanol and carbon dioxide by culturing the engineered bacteria described herein with a source of methanol and carbon dioxide, forming C4-10 fatty acids, harvesting said C4-10 fatty acids. The fatty acids can be used as is, or converted to other desirable compounds such as hydrocarbons or esters.

The fatty acids may themselves be useful, and the final step omitted, but for alkanes the final step would be a chemical reduction to the alkanes. Alternatively other derivatives, such as esters may be desired, and the final step could be an esterification to make e.g., biodiesel.

Preferably, the above bacteria also have reduced fermentation pathways leading to acetate, lactate, ethanol and/or formate. Many such mutants are already available in the art and can be used as host cells, or the vectors can be used to introduce, e.g., knock-out mutations in the host cell. See e.g, ADH⁻, LDH⁻, ACT⁻, PTA⁻, or combined ACK-PTA⁻, See e.g., US20130203137, U.S. Pat. No. 7,709,261, US20060141594, U.S. Pat. No. 7,790,416.

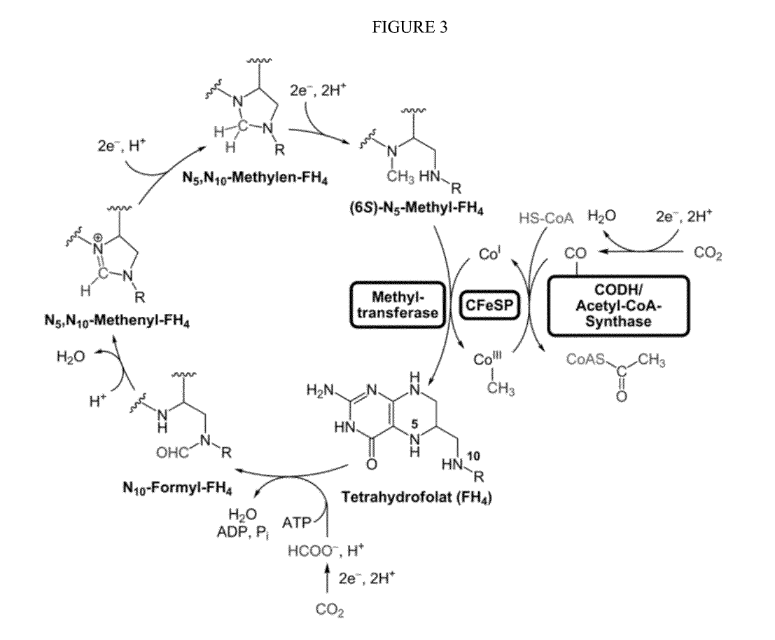

Acetogens may be a useful starting host, as they may contain one or more of the required enzymes (e.g. certain bacteria contain an enzyme for reaction 6), and be suitable for making C4-10 products. Most acetogens use the “Wood-Ljungdahl” pathway. The Wood-Ljungdahl pathway is a set of biochemical reactions used by some bacteria and archaea. It is also known as the reductive acetyl-CoA pathway, and enables certain organisms to use hydrogen as an electron donor and carbon dioxide as an electron acceptor as well as a building block for biosynthesis. In this pathway (see FIG. 3) carbon dioxide is reduced to carbon monoxide, which is then converted to acetyl coenzyme A. Two enzymes participate, CO Dehydrogenase and acetyl-CoA synthase. The former catalyzes the reduction of the CO₂ and the latter combines the resulting CO with a methyl group to give acetyl-CoA. Unlike the Reverse Krebs cycle and the Calvin cycle, this process is not cyclic.

Many acetogens are thought to be strict anaerobes, thus it may be preferred to perform the needed engineering in a more easily grown bacteria, such as E. Coli, or other commonly engineering microbe. However, acetogens are also present in aerated soils and colonize habitats with fluctuating redox conditions (e.g., the rhizosphere of sea grass), suggesting that less strict isolates are obtainable, as confirmed by Mullin's work.

In 1993, Tanner's group isolated a Clostridium ljungdahlii sp. nov. strain ATCC 49587T (T=type strain) from chicken yard waste, and found that this gram-positive, motile, sporeforming rod's metabolism was primarily acetogenic. C. ljungdahlii grew with carbon monoxide, hydrogen and carbon dioxide, ethanol, pyruvate, arabinose, xylose, fructose, or glucose. Methanol, ferulic acid, lactate, galactose, and mannose did not support growth. C. ljungdahlii is the first acetogen in clostridial 23S rRNA homology group I.

The complete genome of Clostridium ljungdahlii DSM 13528 chromosome, is available at NC_{—}014328 (4630065 bp circular DNA), and such information will be very helpful in implementing an efficient genetic engineering plan. This organism might be an advantageous host even though it is an obligate anaerobe because one or more enzymes are already present. Further, oxygen tolerance varies between species, some capable of surviving in up to 8% oxygen, others losing viability unless the oxygen concentration is less than 0.5%, thus it may be possible to screen for O₂ tolerant isolates or select for same by increasing O₂ levels in culture.

Other acetogens include Clostridium autoethanogenum, Eurobacterium limosum, Clostridium carboxidivorans P7, Peptostreptococcus products, and Butyribacterium methylotrophicum.

Still other bacteria that could be useful hosts include Clostridium, Butyrobacterium, Moorella thermoacetica, Sporomusa, Thermacetogenium phaeum, Clostridium thermocellum, Acetogenium kivui, Acetobacterium woodii, Butyribacterium methylotrophicum, Clostridium ljungdahlii, Clostridium thermoautotrophicum, Clostridium tyrobutyricum, or Eubacterium limosum.

Generally speaking we have referenced protein names herein and included EC numbers for accurate identification, but it is understood that a change in protein activity can of course be effected by changing the gene. This provides clarity since the gene nomenclature can be widely divergent in bacteria, but the proteins are defined by their activities and EC numbers.

Once an exemplary protein is obtained, e.g., in E. coli, which is completely sequenced and which is the workhorse of genetic engineering and bio-production, many additional examples proteins of similar activity can be identified by BLAST search or database search. Further, every protein record is linked to a gene record, making it easy to design overexpression vectors. Many of the needed enzymes are already available in vectors, and can often be obtained from cell depositories or from the researchers who cloned them. But, if necessary, new clones can be prepared based on available sequence information using RT-PCR techniques. Thus, it should be easily possible to obtain all of the needed enzymes for overexpression, and in fact, we already have FDH, clones, and are collecting the rest.

Understanding the inherent degeneracy of the genetic code allows one of ordinary skill in the art to design multiple nucleotides that encode the same amino acid sequence. NCBI™ provides codon usage databases for optimizing DNA sequences for protein expression in various species. Using such databases, a gene or cDNA may be “optimized” for expression in E. coli, or other bacterial species using the codon bias for the species in which the gene will be expressed.

In calculating “% identity” the unaligned terminal portions of the query sequence are not included in the calculation. The identity is calculated over the entire length of the reference sequence, thus short local alignments with a query sequence are not relevant (e.g., % identity=number of aligned residues in the query sequence/length of reference sequence). Alignments are performed using BLAST homology alignment as described by Tatusova T A & Madden T L (1999) FEMS Microbiol. Lett. 174:247-250. The default parameters were used, except the filters were turned OFF. As of Jan. 1, 2001 the default parameters were as follows: BLASTN or BLASTP as appropriate; Matrix=none for BLASTN, BLOSUM62 for BLASTP; G Cost to open gap default=5 for nucleotides, 1 1 for proteins; E Cost to extend gap [Integer] default=2 for nucleotides, 1 for proteins; q Penalty for nucleotide mismatch [Integer] default=−3; r reward for nucleotide match [Integer] default=1; e expect value [Real] default=10; W word size [Integer] default=1 1 for nucleotides, 3 for proteins; y Dropoff (X) for blast extensions in bits (default if zero) default=20 for blastn, 7 for other programs; X dropoff value for gapped alignment (in bits) 30 for blastn, 15 for other programs; Z final X dropoff value for gapped alignment (in bits) 50 for blastn, 25 for other programs. This program is available online at NCBI™ (ncbi.nlm.nih.gov/BLAST/).

As used herein the specification, “a” or “an” may mean one or more. As used herein in the claim(s), when used in conjunction with the word “comprising”, the words “a” or “an” may mean one or more than one. As used herein “another” may mean at least a second or more.

The term “about” means the stated value plus or minus the margin of error of measurement or plus or minus 10% if no method of measurement is indicated.

The use of the term “or” in the claims is used to mean “and/or” unless explicitly indicated to refer to alternatives only or if the alternatives are mutually exclusive.

The terms “comprise”, “have”, “include” and “contain” (and their variants) are open-ended linking verbs and allow the addition of other elements when used in a claim.

As used herein, the expressions “cell”, “cell line” and “cell culture” are used interchangeably and all such designations include progeny. Thus, the words “cells” and similar designations include the primary subject cell and cultures derived therefrom without regard for the number of transfers. It is also understood that all progeny may not be precisely identical in DNA content, due to deliberate or inadvertent mutations that arise after genetic engineering is concluded. Mutant progeny that have the same function or biological activity as screened for in the originally transformed cell are included. Where distinct designations are intended, it will be clear from the context.

The terms “operably associated” or “operably linked,” as used herein, refer to functionally coupled nucleic acid sequences.

As used herein “recombinant” is relating to, derived from, or containing genetically engineered material. In other words, the genome was intentionally manipulated in some way.

“Reduced activity” or “inactivation” is defined herein to be at least a 75% reduction in protein activity, as compared with an appropriate control species. Preferably, at least 80, 85, 90, 95% reduction in activity is attained, and in the most preferred embodiment, the activity is eliminated (100%). Proteins can be inactivated with inhibitors, by mutation, or by suppression of expression or translation, and the like.

“Overexpression” or “overexpressed” is defined herein to be at least 150% of protein activity as compared with an appropriate control species. Since many of the relevant proteins are not available in the host species being designed for C1 to C4-10 conversion, one would have to compare against the species of gene/protein origin. Overexpression can be achieved by mutating the protein to produce a more active form or a form that is resistant to inhibition, by removing inhibitors, or adding activators, and the like. Overexpression can also be achieved by removing repressors, adding multiple copies of the gene to the cell, or upregulating the endogenous gene, and the like. An overexpressed protein can be represented by the + symbol, e.g., FDH+.

The terms “disruption” as used herein, refer to cell strains in which the native gene or promoter is mutated, deleted, interrupted, or down regulated in such a way as to decrease the activity of the protein at least 90% over the wild type un-disrupted protein. A gene or protein can be completely (100%) reduced by knockout or removal of the entire genomic DNA sequence. A knockout mutant can be represented by the A symbol.

Use of a frame shift mutation, early stop codon, point mutations of critical residues, or deletions or insertions, and the like, can completely inactivate (100%) gene product by completely preventing transcription and/or translation of active protein.

The following abbreviations, plasmids and strains are used herein:

| ABBREVIATION | FULL NAME |
|---|---|
| ACK | acetate kinase |
| ackA | E. coli gene encoding ACK |
| ADH | Alcohol dehydrogenase |
| adhE | E. coli gene encoding ADH |
| CmR | chloramphenicol resistance gene |
| EtOH | ethanol |
| FDH | Formate dehydrogenase, cofactor dependant |
| LDH | lactate dehydrogenase |
| IdhA | E. coli gene encoding LDH, NAD⁺-dependent |
| PTA | Phosphotransacetylase |
| pta | E. coli gene encoding PTA |

As used herein, “ADH” means a protein having alcohol dehydrogenase activity. Many such proteins are available in GenBank. The E. coli gene encoding this protein is adhE, but it may have other names in other species.

As used herein, “LDH” means a protein having lactate dehydrogenase activity. Many such proteins are available in GenBank. The E. coli gene encoding this protein is ldhA, but it may have other names in other species.

Disruptions in ADH, LDH, ACK-PTA, etc. can be derived as described in U.S. Pat. No. 7,223,567, incorporated herein in its entirety by reference.

In one embodiment the FDH is from Candida boidinii, but obviously any functional FDH can be used from any source since by definition FDH is an NAD+formate dehydrogenase and will catalyze the same reaction. Thus, the FDH can selected from the group consisting of Candida boidinii FDH, Candida methylica FDH, Pseodomonas sp 101 FDH, Arabidopsis thaliana FDH, Staphylococcus aureus FDH, Saccharomyces bayanus FDH, Saccharomyces exiguus FDH, Saccharomyces servazzii FDH, Zygosaccharomyces rouxil FDH, Saccharomyces kluyveri FDH, Kluyveromyces thermotolerans FDH, Kluyveromyces lactis FDH, Kluyveromyces marxianus FDH, Pichia angusta FDH, Debaryomyces hansenii FDH, Pichia sorbitophila FDH, Candida tropicalis FDH, and Yarrowia lipolytica FDH, among others.

Each of the following citations is incorporated by reference herein in its entirety for all purposes:
Loke H K, Bennett G N, Lindahl P A Active acetyl-CoA synthase from Clostridium thermoaceticum obtained by cloning and heterologous expression of acsAB in Escherichia coli. Proc Natl Acad Sci USA. 2000 November 7; 97(23):12530-5.
Al-Hinai M A, Fast A G, Papoutsakis E T Novel system for efficient isolation of Clostridium double-crossover allelic exchange mutants enabling markerless chromosomal gene deletions and DNA integration, Appl Environ Microbiol. 2012 November; 78(22):8112-21.
Kuehne S A, Minton N P, ClosTron-mediated engineering of Clostridium, Bioengineered. 2012 July-August; 3(4):247-54.
Ng Y K, et al., Expanding the repertoire of gene tools for precise manipulation of the Clostridium difficile genome: allelic exchange using pyrE alleles, PLoS One. 2013; 8(2):e56051.
Lee J, et al, Metabolic engineering of Clostridium acetobutylicum ATCC 824 for isopropanol-butanol-ethanol fermentation, Appl Environ Microbiol. 2012 March; 78(5):1416-23.
Leang C, et al., A genetic system for Clostridium ljungdahlii: a chassis for autotrophic production of biocommodities and a model homoacetogen, Lovley D R., Appl Environ Microbiol. 2013 February; 79(4):1102-9.
Bevers, L. E. et al., WOR5, a novel tungsten-containing aldehyde oxidoreductase from Pyrococcus furiosus with a broad substrate specificity; J. Bacteriol. 187, 7056-7061 (2005).
U.S. Pat. No. 7,223,567, US20130203137, U.S. Pat. No. 7,709,261, US20060141594, U.S. Pat. No. 7,790,416.

1. A genetically engineered bacteria, expressing each of:
a. methanol dehydrogenase;
b. methylene tetrahydrofolate reductase;
c. formaldehyde dehydrogenase;
d. formate dehydrogenase;
e. carbon monoxide dehydrogenase-acetyl-CoA synthase;
f. thiolase or acetoacetyl-CoA synthase;
g. hydroxybutyryl-CoA dehydrogenase;
h. crotonase or Enoyl-CoA hydratase-isomerase or peroxisomal bifunctional enzyme;
i. butyryl-CoA dehydrogenase or trans-enoyl-CoA reductase; and
j. phosphotransbutyrylase and butyrate kinase;
wherein said bacteria able to convert methanol and carbon dioxide to ≧C4 fatty acids.

2. The bacteria of claim 1, wherein one or more enzymes a to j are overexpressed.

3. The bacteria of claim 1, wherein said bacteria is an acetogenic bacteria.

4. The bacteria of claim 1, wherein said bacteria is a Clostridium, Clostridium thermocellum, Clostridium ljungdahlii, Clostridium thermoautotrophicum, or Clostridium tyrobutyricum.

5. The bacteria of claim 1, wherein said bacteria is a Butyrobacterium, Moorella thermoacetica, Sporomusa, Thermacetogenium phaeum, Acetogenium kivui, Acetobacterium woodii, or Eubacterium.

6. The bacteria of claim 1, wherein one or more of said enzymes are selected from those listed in Table A or B.

7. A method for the bioproduction of fatty acids from methanol and carbon dioxide by growing the bacteria of claim 1 in a medium comprising methanol and carbon dioxide until fatty acids are produced, and harvesting said produced fatty acids.

8. The method of claim 7, further comprising converting said fatty acids to hydrocarbons.

9. The method of claim 7, further comprising converting said fatty acids to esters.
a. growing the bacteria of claim 1 in a culture medium providing a C1 carbon source;
b. conversion of said C1 carbon source to fatty acids of chain length ≧C4,
c. electrochemical conversion of said fatty acids to hydrocarbons and H₂ and CO₂.

11. The method of claim 10, wherein said H₂ and CO₂ are recycled to said culture medium.

12. A method for the bioproduction of fatty acids or their derivatives, said method comprising:
a. growing the bacteria of claim 2 in a culture medium providing a C1 carbon source;
b. conversion of said C1 carbon source to fatty acids of chain length ≧C4; and
c. isolation of said fatty acids.

13. The method of claim 12, wherein said fatty acids are converted to esters.

14. The method of claim 12, wherein said fatty acids are converted to hydrocarbons.

15. The method of claim 12, followed by electrochemical conversion of said fatty acids to hydrocarbons and H₂ and CO₂.

16. The method of claim 12, wherein said H₂ and CO₂ are recycled to said culture medium.

17. A system for producing fatty acids or hydrocarbons, said system comprising a bioreactor containing a growth medium and a bacteria of claim 2, said bioreactor fluidly coupled to a separator for separating fatty acids from bacteria and/or growth medium.

18. The system of claim 17, further comprising a first flow return pipe fluidly coupling said bioreactor and said separator for returning said bacteria and/or growth medium to said bioreactor.

19. The system of claim 17, wherein said fatty acids are secreted and wherein said separator skims fatty acids from a surface of said growth medium, and said first flow return pipe returns the growth medium to said bioreactor.

20. The system of claim 17, further comprising a reactor fluidly coupled to said separator, said reactor for electrochemical conversion of said fatty acids to hydrocarbons and H₂ and CO₂.

21. The system of claim 17, further comprising a second flow return pipe fluidly coupling said reactor and said bioreactor for returning said CO₂ to said bioreactor.
